(19) United States
(12) Patent Application Publication
(10) Pub. No.: US 2008/0287750 A1
(43) Pub. Date: Nov. 20, 2008

(54) Ergonomic probes

(75) Inventors: Dan Hashimshony, Givat Ada (IL); Gil Cohen, Jerusalem (IL); Iddo Geltner, Herzlia (IL)

Correspondence Address: MARTIN D. MOYNIHAN d/b/a PRTSI, INC., P.O. BOX 16446, ARLINGTON, VA, 22215, US

(73) Assignee: Dune Medical Devices Ltd., Caesaria (IL)

(21) Appl. No.: 11/797,166

(22) Filed: May 1, 2007

Related U.S. Application Data
(63) Continuation-in-part of application No. 11/705,143, filed on Feb. 12, 2007.
Application No. 11/705,143 is a continuation-in-part of application No. 10/965,752, filed on Oct. 18, 2004, now Pat. No. 7,184,824.
Application No. 10/965,752 is a continuation-in-part of application No. 10/035,428, filed on Jan. 4, 2002, now Pat. No. 6,813,515.
Application No. 10/035,428 is a continuation-in-part of application No. PCT/IL2006/000392, filed on Mar. 29, 2006.
Application No. PCT/IL2006/000392 is a continuation-in-part of application No. 10/567,581, filed on Feb. 8, 2006.
Application No. 10/567,581 is a continuation-in-part of application No. 10/558,831, filed on Nov. 29, 2005.
Application No. 10/558,831 is a continuation-in-part of application No. PCT/IL2006/000908, filed on Aug. 6, 2006.
Application No. PCT/IL2006/000908 is a continuation-in-part of application No. 11/350,102, filed on Feb. 9, 2006.
Application No. 11/350,102 is a continuation-in-part of application No. 11/196,732, filed on Aug. 4, 2005.
Application No. 11/196,732 is a continuation-in-part of application No. 11/487,431, filed on Jul. 17, 2006.
Application No. 11/487,431 is a continuation-in-part of application No. 10/891,750, filed on Jul. 15, 2004, now Pat. No. 7,082,325.
Application No. 10/891,750 is a continuation-in-part of application No. 10/298,196, filed on Nov. 18, 2002.

(51) Int. Cl. A61B 5/05; A61B 5/00

(52) U.S. Cl. 600/301; 600/407

(57) ABSTRACT

An ergonomic device for intraoperative tissue characterization is provided, having a gripping handle and a sensor head, arranged with an angle α between them, wherein in absolute value, α>10 degrees, and α may be adjusted in one, two, or three planes. The device may further comprise a light fixture, configured for lighting the tissue, as it is characterized, a marking module, configured for marking the tissue, as it is characterized, a vacuum system, for improved contact between a sensing surface and the tissue, and a transparent frame, which enables an operator to observe the tissue, as it is characterized. The device may be operative with at least one sensor, selected from the group consisting of an optical sensor, an X-ray sensor, an RF sensor, a MW sensor, an infrared thermography sensor, an ultrasound sensor, an MR sensor, an impedance sensor, a temperature sensor, a biosensor, a chemical sensor, a radioactive-emission sensor, a nonirradiative RF sensor, and a mechanical sensor.

### CROSS REFERENCE TO RELATED APPLICATION

This application is being co-filed with a sister U.S. patent application, entitled “Probes, Systems, and Methods for Examining Tissue According to the Dielectric Properties Thereof” (Attorney Docket No. 38281).

This application is also a continuation-in-part of pending U.S. patent application Ser. No. 11/705,143 filed on Feb. 12, 2007, which is a continuation-in-part of U.S. patent application Ser. No. 10/965,752 filed on Oct. 18, 2004, now U.S. Pat. No. 7,184,824 issued on Feb. 27, 2007 and a continuation-in-part of U.S. patent application Ser. No. 10/035,428 filed on Jan. 4, 2002, now U.S. Pat. No. 6,813,515 issued on Nov. 2, 2004.

Additionally, this application is a continuation-in-part of PCT Patent Application No. PCT/IL2006/000392 filed on Mar. 29, 2006, which claims the benefit of U.S. Provisional Patent Application No. 60/665,842 filed on Mar. 29, 2005, now expired.

Additionally, this application is a continuation-in-part of pending U.S. patent application Ser. No. 10/567,581 filed on Feb. 8, 2006, which is a National Phase of PCT Patent Application No. PCT/IL2006/000015 filed on Jan. 4, 2006, which claims the benefit of U.S. Provisional Patent Application No. 60/665,842 filed on Mar. 29, 2005, now expired, and U.S. Provisional Patent Application No. 60/641,081 filed on Jan. 4, 2005, now expired.

Additionally, this application is a continuation-in-part of pending U.S. patent application Ser. No. 10/558,831 filed on Nov. 29, 2005, which is a National Phase of PCT Patent Application No. PCT/IL2005/000330 filed on Mar. 23, 2005, which claims the benefit of U.S. Provisional Patent Application No. 60/555,901 filed on Mar. 23, 2004, now expired.

Additionally, this application is a continuation-in-part of PCT Patent Application No. PCT/IL2006/000908 filed on Aug. 6, 2006, which is a continuation-in-part of pending U.S. patent application Ser. No. 11/350,102 filed on Feb. 9, 2006, and a continuation-in-part of pending U.S. patent application Ser. No. 11/196,732 filed on Aug. 4, 2005.

Additionally, this application is a continuation-in-part of pending U.S. patent application Ser. No. 11/487,431 filed on Jul. 17, 2006, which is a continuation in part of U.S. patent application Ser. No. 10/891,750 filed on Jul. 15, 2004, now U.S. Pat. No. 7,082,325 issued on Jul. 25, 2006.

Additionally, this application is a continuation-in-part of pending U.S. patent application Ser. No. 10/298,196 filed on Nov. 18, 2002.

Additionally, this application is a continuation-in-part of pending PCT Patent Application No. PCT/IL2007/000071 filed on Jan. 18, 2007, which claims the benefit of U.S. Provisional Patent Application No. 60/759,555 filed on Jan. 18, 2006.

The disclosures of all of these are incorporated herein by reference.

### FIELD AND BACKGROUND OF THE INVENTION

The present invention relates to an ergonomic device for intraoperative tissue characterization.

A large number of techniques and sensors are available today for tissue characterization, for example, to determine the presence of abnormal tissue, such as cancerous or pre-cancerous tissue. These may be incorporated into hand-held probes and may be used during operation, to characterize the tissue, in order to determine which portions, if any, need be removed. While the operating principles of different tissue characterization sensors differ, their manipulation, in the hands of a surgeon, is generally similar.

### SUMMARY OF THE INVENTION

An ergonomic device for intraoperative tissue characterization is provided, having a gripping handle and a sensor head, arranged with an angle α between them, wherein in absolute value, α>10 degrees, and a may be adjusted in one, two, or three planes. The device may further comprise a light fixture, configured for lighting the tissue, as it is characterized, a marking module, configured for marking the tissue, as it is characterized, a vacuum system, for improved contact between a sensing surface and the tissue, and a transparent frame, which enables an operator to observe the tissue, as it is characterized. The device may be operative with at least one sensor, selected from the group consisting of an optical sensor, an X-ray sensor, an RF sensor, a MW sensor, an infrared thermography sensor, an ultrasound sensor, an MR sensor, an impedance sensor, a temperature sensor, a biosensor, a chemical sensor, a radioactive-emission sensor, a nonirradiative RF sensor, and a mechanical sensor.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 schematically illustrates an ergonomic device for intraoperative tissue characterization, designed with a sensor head, at an angle to a gripping handle, in accordance with an embodiment of the present invention;

FIG. 2 schematically illustrates the device of FIG. 1, wherein the angle is adjustable, in accordance with an embodiment of the present invention;

FIGS. 3A-3C schematically illustrate manners of varying the angle of the device of FIG. 2, in accordance with embodiments of the present invention;

FIG. 4 schematically illustrates light fixtures, associated with an ergonomic device for intraoperative tissue characterization, in accordance with an embodiment of the present invention;

FIGS. 5A and 5B schematically illustrate light fixtures, associated with an ergonomic device for intraoperative tissue characterization, in accordance with another embodiment of the present invention;

FIGS. 6A-6C schematically illustrate a vacuum system, associated with an ergonomic device for intraoperative tissue characterization, for improving contact between a sensing surface and the tissue for characterization, by suction, in accordance with an embodiment of the present invention;

FIGS. 7A-7C schematically illustrate a marking module, associated with an ergonomic device for intraoperative tissue characterization, for marking the characterized region of the tissue, in accordance with an embodiment of the present invention; and

FIGS. 8A and 8B schematically illustrate control systems for an ergonomic device for intraoperative tissue characterization, in accordance with embodiments of the present invention.

### DESCRIPTION OF THE PREFERRED EMBODIMENTS

The present invention relates to an ergonomic device for intraoperative tissue characterization, having a gripping handle and a sensor head, arranged with an angle α between them, wherein in absolute value, α>10 degrees, and a may be adjusted in one, two, or three planes. The device may further comprise a light fixture, configured for lighting the tissue, as it is characterized, a marking module, configured for marking the tissue, as it is characterized, a vacuum system, for improved contact between a sensing surface and the tissue, and a transparent frame, which enables an operator to observe the tissue, as it is characterized. The device may be operative with at least one sensor, selected from the group consisting of an optical sensor, an X-ray sensor, an RF sensor, a MW sensor, an infrared thermography sensor, an ultrasound sensor, an MR sensor, an impedance sensor, a temperature sensor, a biosensor, a chemical sensor, a radioactive-emission sensor, a nonirradiative RF sensor, and a mechanical sensor.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The principles and operation of the device for tissue-characterization, according to some embodiments of the present invention, may be better understood with reference to the drawings and accompanying descriptions.

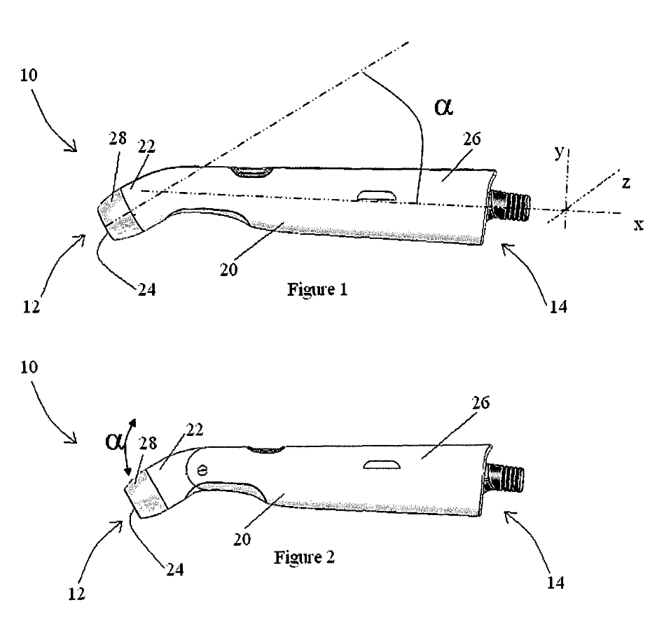

Referring now to the drawings, FIG. 1 schematically illustrates an ergonomic device 10 for intraoperative tissue characterization, designed with a sensor head, at an angle to a gripping handle, in accordance with an embodiment of the present invention. The ergonomic device 10 includes:

a body 20, which defines proximal and distal portions, 12 and 14, with respect to the tissue, along a longitudinal axis, in an x direction;
a gripping handle 26, at the distal portion 14; and
a sensor head 22, at the proximal portion 12, the sensor head 22 comprising at least one sensor 24 for tissue characterization,

wherein the gripping handle 26 and the sensor head 22 are arranged at an angle α, wherein in absolute value, α>10 degrees. The angle α may be in the x;y plane, the x;z plane, or the y:z plane. It will be appreciated that the angle α may be a solid angle, with components in any one of the x;y, x;z and y:z planes.

The purpose of the angle α is to provide better handling and control of the device 10 by an operator (not shown).

Referring further to the drawings, FIG. 2 schematically illustrates the device 10 of FIG. 1, wherein the angle α is adjustable, in accordance with an embodiment of the present invention. Preferably, the angle α may be adjusted, by the operator, either before the examination begins, or during the examination, and may further include a setting of 0 degrees. Furthermore, the angle α may be adjusted in any one of the x;y, x;z and y:z planes, or in two planes, or in all three planes, that is along one, two, or three axes of rotation.

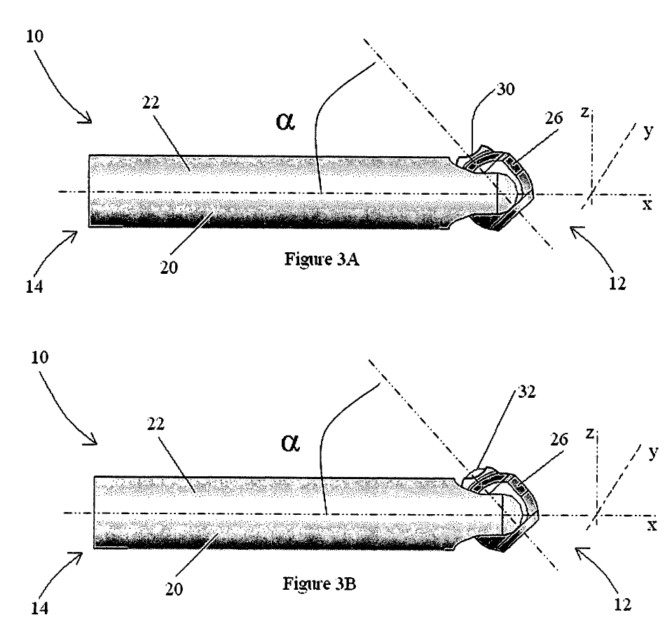

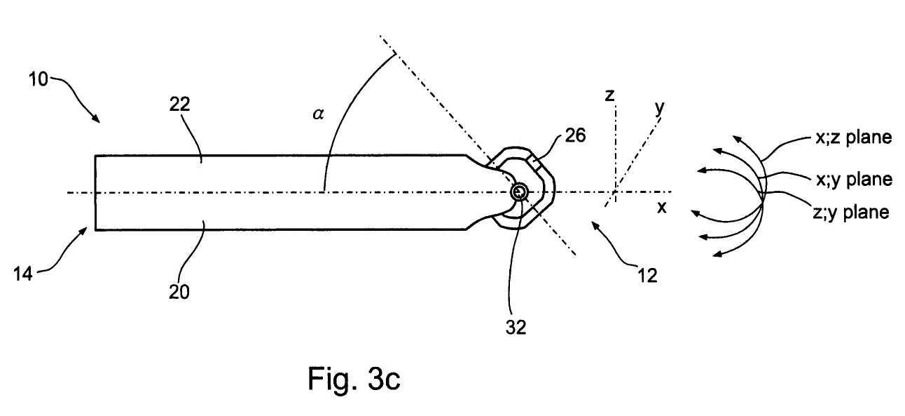

Referring further to the drawings, FIGS. 3A-3C schematically illustrate manners of varying the angle of the device of FIG. 2, in accordance with an embodiment of the present invention.

As seen in FIG. 3A, control of the angle α is provided by an indentation 30, in the sensor head 22. The indentation 30 is configured for finger control, by the operator. A locking feature or a stop may be provided, as well.

As seen in FIG. 3B, control of the angle α is provided by a friction pad 32, in the sensor head 22. The friction pad 32 is also configured for finger control, by the operator. The friction pad 32 may operate in a manner similar to a roller of a computer mouse. Again, a locking feature or a stop may be provided.

As seen in FIG. 3C, control of the angle α is provided by the friction pad 32, configured for finger control, for rotation in two or in three planes, of the x;y, x;z, and z;y planes.

Returning to FIGS. 1 and 2, they further illustrate the sensor head 22 with a transparent frame 28, for providing better visibility of the tissue region that is being characterized, in accordance with embodiments of the present invention.

It may happen that the construction of the sensor head 22 is such as to obstruct a view of the tissue that is being characterized from the operator. The transparent frame 28 remedies that situation, by providing visibility through the frame.

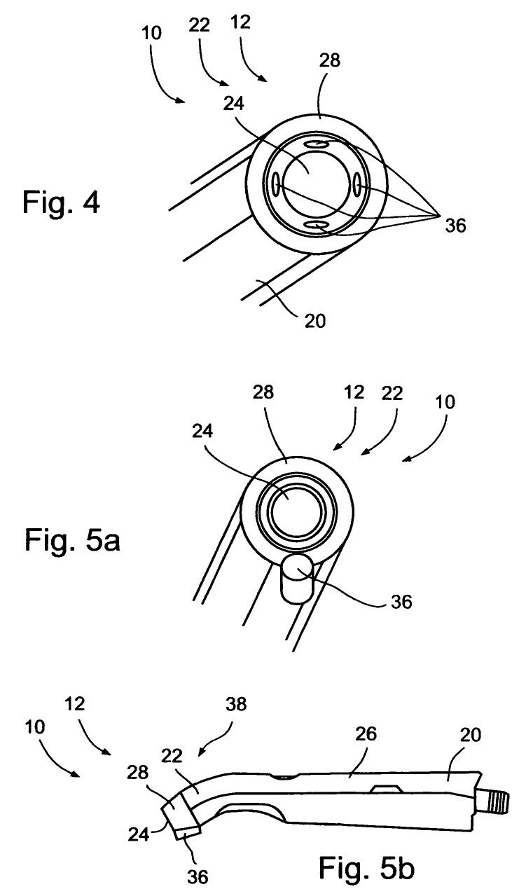

Referring further to the drawings, FIG. 4 schematically illustrates light fixtures, associated with an ergonomic device for intraoperative tissue characterization, in accordance with an embodiment of the present invention.

Accordingly, light fixtures 36 may be arranged on the proximal surface of the sensor head 22, for example, between the transparent frame 28 and the sensor 24. It will be appreciated that a single light fixture 36 may be used. Alternatively, several light fixtures 36 may be employed.

Referring further to the drawings, FIGS. 5A and 5B schematically illustrate light fixtures, associated with an ergonomic device for intraoperative tissue characterization, in accordance with another embodiment of the present invention.

Accordingly, the light fixtures 36 may be placed on the external side of the frame 28. The operator (not shown) defines an operator end 38, and preferably, the light fixture 36 is placed at about 180 degrees from the operator end 38, so as not to obstruct the operator's view.

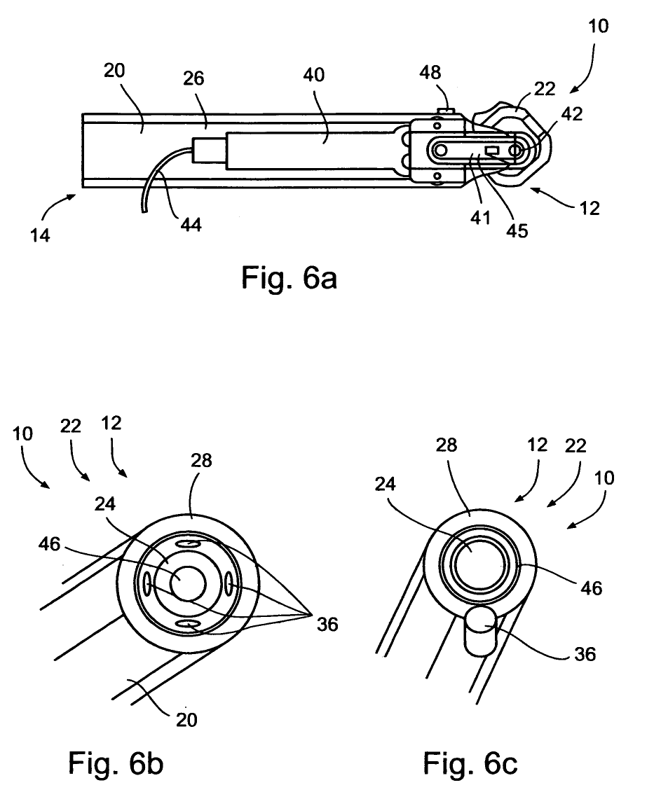

Referring further to the drawings, FIGS. 6A-6C schematically illustrate a vacuum system, associated with an ergonomic device for intraoperative tissue characterization, for improving contact between a sensing surface and the tissue for characterization, by suction, in accordance with an embodiment of the present invention.

Accordingly, a vacuum system 40, having a vacuum source 44 may be incorporated within the device 10. The vacuum system 40 may further include a feature that enables adjustment of the angle α (FIGS. 1-3C). Thus, the vacuum system 40 may include a vacuum communication conduit 45, comprising first and second parts 41 and 42. The first part 41 is aligned with the handle 26, while the second part 42 is integrated into the axis of rotation of the sensing head 22 and includes sealing components, such as “o” rings. Thus vacuum is maintained even as the angle α between the sensing head 22 and the gripping handle 26 is adjusted.

Alternatively, the vacuum communication between the sensing head 22 and the gripping handle 26 may be constructed as a bellow, or as flexible tubing.

A vacuum nozzle 46 may be at the center of the sensor 24, as seen in FIG. 6B, or around it, as seen in FIG. 6C.

The vacuum enables the operator to ensure good and effective contact between the sensor 24 and the tissue for characterization.

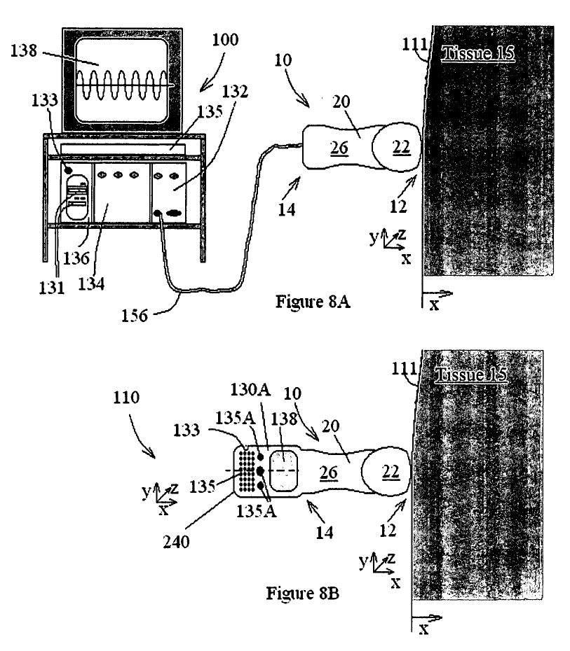

The vacuum may be applied from a control knob 48 on the gripping handle 26 (FIG. 6A), or from the systems 100 or 110, illustrated hereinbelow, in conjunction with FIGS. 8A and 8B.

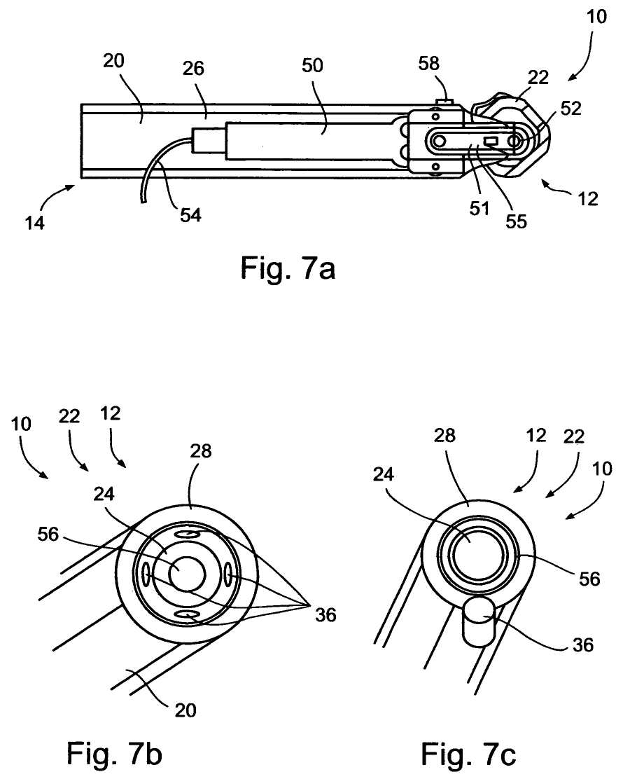

Referring further to the drawings, FIGS. 7A-7C schematically illustrate a marking module, associated with an ergonomic device for intraoperative tissue characterization, for marking the characterized region of the tissue, in accordance with an embodiment of the present invention.

A marking module 50 may be constructed in a manner analogous to the vacuum system 40, having a marking-material source 54 and a marking communication conduit 55, of two parts 51 and 52. The first part 51 is aligned with the handle 26, while the second part 52 is integrated into the axis of rotation of the sensing head 22 and includes sealing components, such as “o” rings. Thus marking communication is maintained even as the angle α between the sensing head 22 and the gripping handle 26 is adjusted.

Alternatively, the marking communication between the sensing head 22 and the gripping handle 26 is constructed as a bellow, or as flexible tubing.

A jet nozzle 56, from which the marking material issues, may be at the center of the sensor 24, as seen in FIG. 7B, or peripheral to it, as seen in FIG. 7C.

The operator may control the marking from a control knob 58 on the gripping handle 26 (FIG. 7A), or from the systems 100 or 110, illustrated hereinbelow, in conjunction with FIGS. 8A and 8B.

Marking in situ is intended to identify the tissue that was characterized, when the operator wishes to return to it, for example, for a second examination, for taking a biopsy sample, or for performing an operation. The marking physically marks a tissue associated with certain detection results of the device 10.

The marking material may be, a visually detectable substance, e.g., a three color biological marking ink, emitted from the jet nozzle 56. The jet nozzle may be controlled from the gripping handle 26 or from the control and instrumentation system 100 (FIG. 1).

Other forms of detectable marking material can be, for example, a physical marker conjugated to antibodies, metal balls, IR paint, etc. The marker can also be a solid marker like a small metal pin, or a combination of solid balls painted with a distinguishing color. The solid balls are palpable and the color is visible. The marker can also be detectable by other known modalities, like X-ray or ultrasound.

Referring further to the drawings, FIGS. 8A and 8B schematically illustrate a control and instrumentation systems for the ergonomic device for intraoperative tissue characterization, in accordance with embodiments of the present invention.

As seen in FIG. 8A, a control and instrumentation system 100 of the ergonomic device 10 includes a signal generator 132, an analyzer 134, and a controller 136, although these may be integrated into a single unit. A user interface may be provided, for example, in the form of read and write drives 131, such as, a diskette, a CD, a DVD, a disk-on-key and the like, for providing predetermined operating parameters and settings, and in order to store test results. A display screen 138 may display the resonating response. It will be appreciated that other output means, for example, a printer or a facsimile, are also possible. A keyboard 135 may be used to input data such as patient details, date and time of a particular test, signal parameters, and the like. Additionally, the controller 136 may include other input and output devices, for example, a USB port 133, and other features, as known.

Alternatively, as seen in FIG. 8B, a control and instrumentation system 110 may be integrated with the ergonomic device 10, and may further include additional control knobs 135A, for example, for controlling the vacuum system 40 or the marking module 50.

The sensor 24 may be an optical sensor, an X-ray sensor, an RF sensor, a MW sensor, an infrared thermography sensor, an ultrasound sensor, an MR sensor, an impedance sensor, a temperature sensor, a biosensor, a chemical sensor, a radioactive-emission sensor, a mechanical sensor, and (or) a nonirradiative RF sensor. It will be appreciated that two or more sensors may be employed, and they may be of a same kind or of different kinds.

Tissue characterization by ultrasonography: Ultrasonography is a medical imaging technique, using high frequency sound waves in the range of about 1 to 40 MHz and their echoes. The sound waves travel in the body and are reflected by interfaces between different types of tissues, such as between a healthy tissue and a denser cancerous tissue, or between a portion of a soft tissue and a bone. The ultrasound probe receives the reflected sound waves and the associated instrumentation calculates the distances from the probe to the reflecting boundaries.

The ultrasound probe includes a piezoelectric crystal, which produces an electric signal in response to a pressure pulse. The shape of the probe determines its field of view, and the frequency of the emitted sound determines the minimal detectable object size. Generally, the probes are designed to move across the surface of the body. However, some probes are designed to be inserted through body lumens, such as the vagina or the rectum, so as to get closer to the organ being examined.

Before the early 1970's ultrasound imaging systems were able to record only the strong echoes arising from the outlines of an organ, but not the low-level echoes of the internal structure. In 1972 a refined imaging mode was introduced called gray-scale display, in which the internal texture of many organs became visible. In consequence, ultrasound imaging became a useful tool for imaging tumors, for example, in the liver.

A development of recent years is a 3D ultrasound imaging, in which, several two-dimensional images are acquired by moving the probes across the body surface or by rotating probes, inserted into body lumens. The two-dimensional scans are then combined by specialized computer software to form 3D images.

In multiple-element probes, each element has a dedicated electric circuit, so that the beam can be “steered” by changing the timing in which each element sends out a pulse. By sequentially stimulating each element, the beams can be rapidly steered from the left to right, to produce a two-dimensional cross sectional image. Additionally, transducer-pulse controls allow the operator to set and change the frequency and duration of the ultrasound pulses, as well as the scan mode of the machine. A probe formed of array transducers has the ability to be steered as well as focused.

Contrast agents may be used in conjunction with ultrasound imaging, for example as taught by U.S. Pat. No. 6,280,704, to Schutt, et al., entitled, “Ultrasonic imaging system utilizing a long-persistence contrast agent,” whose disclosure is incorporated herein by reference.

Tissue characterization by its dielectric properties: There are several known techniques for local tissue characterization by the tissue's electromagnetic properties.

Commonly owned U.S. Pat. No. 6,813,515, to Hashimshony, entitled, “Method and system for examining tissue according to the dielectric properties thereof,” whose disclosure is incorporated herein by reference, describes a method and system for examining tissue in order to differentiate it from other tissue, according to the dielectric properties of the examined tissue. The method includes applying an electrical pulse to the tissue to be examined via a probe formed with an open cavity such that the probe generates an electrical fringe field in examined tissue within the cavity and produces a reflected electrical pulse therefrom with negligible radiation penetrating into other tissues or biological bodies near the examined tissue; detecting the reflected electrical pulse; and comparing electrical characteristics of the reflected electrical pulse with respect to the applied electrical pulse to provide an indication of the dielectric properties of the examined tissue.

Furthermore, commonly owned U.S. Patent Application 60/641,081, entitled, “Device and Method for Tissue Characterization in a Body Lumen, by an Endoscopic Electromagnetic Probe,” whose disclosure is incorporated herein by reference, discloses a device and method for tissue characterization in a body lumen, for the detection of abnormalities, using an electromagnetic probe, mounted on an endoscope. The endoscope may be designed for insertion in a body lumen, selected from the group consisting of an oral cavity, a gastrointestinal tract, a rectum, a colon, bronchi, a vagina, a cervix, a urinary tract, and blood vessels. Additionally, it may be designed for insertion in a trocar valve.

Additionally, commonly owned U.S. Patent Application 60/665,842, entitled, “Electromagnetic Sensors for tissue Characterization,” whose disclosure is incorporated herein by reference, discloses a sensor, comprising: a resonating element, formed as a conductive structure, configured to be placed proximally to an edge of a tissue for characterization, without penetrating the tissue, and having a diameter-equivalent D, which defines a cross-sectional area of the resonating element, on a plane substantially parallel with the edge; and at least one conductive lead, for providing communication with an external system, wherein the resonating element is configured to resonate at a free-air wavelength range of between about λ and about 10λ, wherein λ is at least about ten times the diameter-equivalent D, and wherein upon receiving a signal in the range of between about λ and about 10λ, the sensor is configured to induce electric and magnetic fields, in a near zone, in the tissue, the near zone being a hemisphere having a diameter of substantially D, beginning with the edge, while causing negligible radiation in a far zone, so that the tissue, in the near zone, effectively functions as part of the resonating element, varying a resonating response to the sensor, and so the tissue, in the near zone, is thereby characterized by its electromagnetic properties, by the resonating response to the sensor.

Commonly owned applications and patents, which relate to tissue characterization by dielectric properties, include:

pending U.S. patent application Ser. No. 10/965,752, filed on Oct. 18, 2004 [our 28734—US—systems and methods for examining tissue according to the dielectric properties], which is a continuation of U.S. patent application Ser. No. 10/035,428, filed on Jan. 4, 2002, now U.S. Pat. No. 6,813,515, issued on Nov. 2, 2004 [our 22801 US—systems and methods for examining tissue according to the dielectric properties], pending PCT Patent Application No. PCT/IL2006/000392, filed on Mar. 29, 2006 [our 31806 PCT—sensor patent], which claims the benefit of U.S. Provisional Patent Application No. 60/665,842, filed on Mar. 29, 2005, now expired. [our 28988 US Provisional—sensor patent],

pending U.S. patent application Ser. No. 10/567,581, filed on Feb. 8, 2006 [our 30479 US—endoscopic patent] which is a National Phase of PCT Patent Application No. PCT/IL2006/000015, filed on Jan. 4, 2006 [our 30480 PCT—endoscopic patent], which claims the benefit of U.S. Provisional Patent Application No. 60/665,842, filed on Mar. 29, 2005, now expired, [our 28988 US Provisional—sensor patent] and U.S. Provisional Patent Application No. 60/641,081, filed on Jan. 4, 2005, now expired. [our 28959 US Provisional—endoscopic patent].

The contents of all these pending applications, published applications, and patents is incorporated herein by reference.

In accordance with the present invention, tissue characterization by dielectric properties may involve an RF sensor, a MW sensor, an impedance sensor, or a resonating sensor.

Tissue Characterization by electrical impedance imaging: Electrical impedance imaging relates to measuring the impedance between a point on the surface of the skin and some reference point on the body of a patient. Sometimes, a multi-element probe, formed as a sheet having an array of electrical contacts, is used for obtaining a two-dimensional impedance map of the tissue, for example, the breast. The two-dimensional impedance map may be used, possibly in conjunction with other data, such as mammography, for the detection of cancer.

Rajshekhar, V. (“Continuous impedance monitoring during CT-guided stereotactic surgery: relative value in cystic and solid lesions,” Rajshekhar, V., British Journal of Neurosurgery, 1992, 6, 439-444) describes using an impedance probe with a single electrode to measure the impedance characteristics of lesions. The objective of the study was to use the measurements made in the lesions to determine the extent of the lesions and to localize the lesions more accurately. The probe was guided to the tumor by CT and four measurements were made within the lesion as the probe passed through the lesion. A biopsy of the lesion was performed using the outer sheath of the probe as a guide to position, after the probe itself was withdrawn.

U.S. Pat. No. 4,458,694, to Sollish, et al., entitled, “Apparatus and method for detection of tumors in tissue,” whose disclosure is incorporated herein by reference, relates to an apparatus for detecting tumors in human breast, based on the dielectric constants of localized regions of the breast tissue. The apparatus includes a probe, including a plurality of elements. The apparatus further includes means for applying an AC signal to the tissue, means for sensing dielectric properties at each of the probe elements at different times, and signal processing circuitry, coupled to the sensing means, for comparing the dielectric properties sensed at the different times. The apparatus thus provides an output of the dielectric constants of localized regions of breast tissue associated with the probe.

Similarly, U.S. Pat. No. 4,291,708 to Frei, et al., entitled, “Apparatus and method for detection of tumors in tissue,” whose disclosure is incorporated herein by reference, relates to apparatus for detecting tumors in human breast tissue, by the dielectric constants of a plurality of localized regions of human breast tissue.

U.S. Pat. Nos. 6,308,097, 6,055,452 and 5,810,742, to Pearlman, A. L., entitled, “Tissue characterization based on impedance images and on impedance measurements,” whose disclosures are incorporated herein by reference, describe apparatus for aiding in the identification of tissue type for an anomalous tissue in an impedance image. The device comprises: means for providing a polychromic emmitance map of a portion of the body; means for determining a plurality of polychromic measures from one or both of a portion of the body; and a display of an indication based on the plurality of polychromic measures.

Tissue Characterization by Optical Fluorescence Spectroscopy: When a sample of large molecules is irradiated, for example, by laser light, it will absorb radiation, and various levels will be excited. Some of the excited states will return back substantially to the previous state, by elastic scattering, and some energy will be lost in internal conversion, collisions and other loss mechanisms. However, some excited states will create fluorescent radiation, which, due to the distribution of states, will give a characteristic wavelength distribution.

Some tumor-marking agents give well-structured fluorescence spectra, when irradiated by laser light. In particular, hematoporphyrin derivatives (HPD), give a well-structured fluorescence spectrum, when excited in the Soret band around 405 nm. The fluorescence spectrum shows typical peaks at about 630 and 690 nm, superimposed in practice on more unstructured tissue autofluorescence. Other useful tumor-marking agents are dihematoporphyrin ether/ester (DHE), hematoporphyrin (HP), polyhematoporphyrin ester (PHE), and tetrasulfonated phthalocyanine (TSPC), when irradiated at 337 nm (N₂ laser).

U.S. Pat. No. 5,115,137, to Andersson-Engels, et al, entitled, “Diagnosis by means of fluorescent light emission from tissue,” whose disclosure is incorporated herein by reference, relates to improved detection of properties of tissue by means of induced fluorescence of large molecules. The tissue character may then be evaluated from the observed large-molecule spectra. According to U.S. Pat. No. 5,115,137, the spectrum for tonsil cancer is clearly different from normal mucosa, due to endogenous porphyrins.

U.S. Pat. No. 6,258,576, to Richards-Kortum, et al., entitled, “Diagnostic method and apparatus for cervical squamous intraepithelial lesions in vitro and in vivo using fluorescence spectroscopy,” whose disclosure is incorporated herein by reference, relates to the use of multiple illumination wavelengths in fluorescence spectroscopy for the diagnosis of cancer and precancer, for example, in the cervix. In this manner, it has been possible to (i) differentiate normal or inflamed tissue from squamous intraepithelial lesions (SILs) and (ii) differentiate high grade SILs from non-high grade SILs. The detection may be performed in vitro or in vivo. Multivariate statistical analysis has been employed to reduce the number of fluorescence excitation-emission wavelength pairs needed to re-develop algorithms that demonstrate a minimum decrease in classification accuracy. For example, the method of the aforementioned patent may comprise illuminating a tissue sample with electromagnetic radiation wavelengths of about 337 nm, 380 nm and 460 nm, to produce fluorescence; detecting a plurality of discrete emission wavelengths from the fluorescence; and calculating from the emission wavelengths a probability that the tissue sample belongs in particular tissue classification.

Commonly owned U.S. Patent Application 2003/01383786, to Hashimshony, entitled, “Method and apparatus for examining tissue for predefined target cells, particularly cancerous cells, and a probe useful for such method and apparatus,” whose disclosure is incorporated herein by reference, teaches a method apparatus and probe for examining tissue and characterizing its type according to measured changes in optical characteristics of the examined tissue. In a preferred embodiment of this method the tissue to be examined is subject to a contrast agent containing small particles of a physical element conjugated with a biological carrier selectively bindable to the target cells. Additionally, energy pulses are applied to the examined tissue, and the changes in impedance and/or the optical characteristics produced by the applied energy pulses are detected and utilized for determining the presence of the target cells in the examined tissue. Furthermore, in a preferred embodiment, the applied energy pulses include laser pulses, and the physical element conjugated with a biological carrier is a light-sensitive semiconductor having an impedance which substantially decrease in the presence of light. Moreover, the same probe used for detecting the targeted cells, may also be used for destroying the cells so targeted.

Tissue Characterization by Optical reflectance spectroscopy: The application optical reflectance spectroscopy for tissue characterization is described, for example, in http://www.sbsp-limb.nichd.nih.gov/html/spectroscopy.html, downloaded on Mar. 15, 2005, disclosing an optical reflectance spectroscopy (ORS) device for measuring the thickness of the epithelial layer, and an evaluation technique based on oblique angle reflectance spectroscopy, that allows assessment of the scattering and absorption properties of the epithelium and stroma, thus providing information on chronic oral epithelial tissue inflammation, which is considered a potential diagnostic precursor to oral cancer.

Additionally, Tomatis, A., et al, studied reflectance images of 43 pigmented lesions of the skin (18 melanomas, 17 common melanocytic naevi and eight dysplastic naevi). Reflectance images were acquired by a telespectrophotometric system and were analyzed in the spectral range from 420 to 1040 nm, to discriminate melanoma from benign melanocytic entities. Different evaluations were carried out considering the whole spectrum, the visible and the near infrared. A total of 33 (76.7%) lesions were correctly diagnosed by the telespectrophotometric system, compared with 35 (81.4%) correct clinical diagnoses. Reflectance in the infrared band appears diagnostically relevant.

Tissue Characterization by Magnetic Resonance (MR): Magnetic resonance is based on the absorption and emission of energy in the radio frequency range of the electromagnetic spectrum, by nuclei having unpaired spins. Magnetic Resonance Imaging (MRI) is based on the imaging of the absorption and emission of energy in the radio frequency range of the electromagnetic spectrum, by nuclei having unpaired spins.

Conventional MRI is a large-apparatus, for whole body imaging, having:

i. a primary magnet, which produces the Bₒ field for the imaging procedure;

ii. gradient coils for producing a gradient in Bₒ;

iii. an RF coil, for producing the Bₗ; magnetic field, necessary to rotate the spins by 90° or 180° and for detecting the MR signal; and

iv. a computer, for controlling the components of the MR imager.

Generally, the magnet is a large horizontal bore superconducting magnet, which provides a homogeneous magnetic field in an internal region within the magnet. A patient or object to be imaged is usually positioned in the homogeneous field region located in the central air gap for imaging. A typical gradient coil system comprises an anti-Helmholtz type of coil. These are two parallel ring shaped coils, around the z axis. Current in each of the two coils flows in opposite directions creating a magnetic field gradient between the two coils.

The RF coil creates a B1 field, which rotates the net magnetization in a pulse sequence. The RF coils may be: 1) transmit and receive coils, 2) receive only coils, and 3) transmit only coils.

As described hereinabove, the MRI relies on a magnetic field in an internal region within the magnet. As such, it is unsuitable as a handheld probe or an endoscopic probe, because the tissue to be imaged has to be in the internal region of the imager,

However, U.S. Pat. No. 5,572,132, to Pulyer, et al., entitled, “MRI probe for external imaging,” whose disclosure is incorporated herein by reference, describes an MRI spectroscopic probe having an external background magnetic field B0 (as opposed to the internal background magnetic filed of the large horizontal bore superconducting magnet). Thus, an MRI catheter for endoscopical imaging of tissue of the artery wall, rectum, urinal tract, intestine, esophagus, nasal passages, vagina and other biomedical applications may be constructed. The probe comprises (i) a miniature primary magnet having a longitudinal axis and an external surface extending in the axial direction, and (ii) a RF coil surrounding and proximal to the surface. The primary magnet is structured and configured to provide a symmetrical, preferably cylindrically shaped, homogeneous field region external to the surface of the magnet. The RF coil receives NMR signals from excited nuclei. For imaging, one or more gradient coils are provided to spatially encode the nuclear spins of nuclei excited by an RF coil, which may be the same coil used for receiving NMR signals or another RF coil.

Additionally, commonly owned US Patent Application 2005/0021019 to Hashimshony et al., entitled “Method and apparatus for examining substance, particularly tissue, to characterize its type,” whose disclosure is incorporated herein by reference, describes a method and apparatus for examining a substance volume to characterize its type, by: applying a polarizing magnetic field through the examined substance: applying RF pulses locally to the examined substance volume such as to invoke electrical impedance (EI) responses signals corresponding to the electrical impedance of the substance, and magnetic resonance (MR) responses signals corresponding to the MR properties of the substance; detecting the EI and MR response signals; and utilizing the detected response signals for characterizing the examined substance volume type.

Contrast agents may be used in conjunction with MRI. For example, U.S. Pat. No. 6,315,981 to Unger, entitled, “Gas filled microspheres as magnetic resonance imaging contrast agents,” whose disclosure is incorporated herein by reference, describes the use of gas filled microspheres as contrast agents for MRI.

Additionally, U.S. Pat. No. 6,747,454, to Belt, entitled, “Array of coils for use in imaging the vasculature of a patient,” whose disclosure is incorporated herein by reference, describes an array of coils, configured for use in imaging the vasculature of a patient.

Furthermore, U.S. Pat. No. 6,677,755, to Belt, et al., “Circuit for selectively enabling and disabling coils of a multi-coil array,” whose disclosure is incorporated herein by reference, describes a circuit, used to selectively enable and disable n-coils. The circuit includes n-drivers powered by a current source. Each n-driver includes a pair of FETs disposed such that a gate of one FET is connected to a gate of the other FET to form a common gate node thereat. The n-drivers are disposed in a totem-pole configuration. The one FET of a first of the n-drivers has (A) a drain linked to a ground and to an end of a first of the n-coils and (B) a source linked to a drain of the one FET of a second of the n-drivers and to an end of a second of the n-coils. The other FET of the first of the n-drivers has (A) a source linked to an opposite end of the first of the n-coils and (B) a drain linked to the end of the second of the n-coils and to the source of the one FET of the first of the n-drivers. The one FET of the second of the n-drivers also has a source linked to a drain of the one FET of a next of the n-drivers and to an end of a next of the n-coils. The other FET of the second of the n-drivers also has (A) a source linked to an opposite end of the second of the n-coils and (B) a drain linked to the end of the next of the n-coils and to the source of the one FET of the second of the n-drivers. This continues until the one FET and the other FET of an nth of the n-drivers are likewise disposed in the totem-pole configuration of the n-drivers, with a source and a drain of the one FET and the other FET, respectively, of the nth of the n-drivers being connected to the current source. Each of the n-drivers is used to operate a corresponding one of the n-coils by being responsive at its common gate node (i) a coil disable signal by activating the one FET thereof and deactivating the other FET thereof thereby not only drawing current away from and thus disabling the corresponding coil but also allowing the current to flow through the one FET and thus be available as a source of current to a successive one of the n-drivers and (ii) a coil enable signal by deactivating the one FET thereof and activating the other FET thereof thereby allowing the current not only to flow serially through the corresponding coil and the other FET thus enabling the corresponding coil but also to be available as a source of current to the successive one of the n-drivers.

Tissue Characterization by Magnetic Resonance Spectroscopy (MRS): In MRS, spectroscopic NMR data is obtained from the examined area. Thus the biochemical information obtained from MRS can be interpreted in relation to a defined anatomical location, and images of metabolite distributions can be generated. MRS can be used to identify surrogate biochemical markers of cellular transformation, thus differentiating benign tumors from malignant, and identifying different tumor types. Prognostic and diagnostic information is derived from the spectrum of malignant tumors. (Breast Cancer Res. 2001, 3:36-40.)

Tissue Characterization by radioactive emission: Radioactive-emission imaging relies on the fact that in general, pathologies, such as malignant tumors and inflammations, display a level of activity different from that of healthy tissue. Thus, radiopharmaceutical, which circulate in the blood stream, are picked up by the active pathologies to a different extent than by the surrounding healthy tissue; in consequence, the pathologies are operative as radioactive-emission sources and may be detected by radioactive-emission imaging.

The pathological feature may appear as a concentrated source of high radiation, or a hot region, as may be associated with a tumor, or as a region of low-level radiation, which is nonetheless above the background level, as may be associated with carcinoma. Additionally, a reversed situation is possible. Dead tissue has practically no pick up of radiopharmaceuticals, and is thus operative as a region of little radiation, or a cold region, below the background level.

Thus radiopharmaceuticals may be used for identifying active pathologies as well as dead tissue, and the image that is constructed is generally termed, a functional image.

The mechanism of localization of a radiopharmaceutical depends on various processes in the organ of interest, such as antigen-antibody reactions, physical trapping of particles, receptor site binding, removal of intentionally damaged cells from circulation, and transport of a chemical species across a cell membrane and into the cell by a normally operative metabolic process. A summary of the mechanisms of localization by radiopharmaceuticals is found in http://www.lunis.luc.edu/nucmed/tutorial/radpharm/i.htm.

The particular choice of a radionuclide for labeling antibodies depends upon the chemistry of the labeling procedure and the isotope nuclear properties, such as, the number of gamma rays emitted, their respective energies, the emission of other particles, such as beta or positrons, the isotope half-life, and the existence of different isotopes of identical chemistry but different half-lives (e.g., I¹³¹ and I¹³³). The usual preferred emission for medical applications is that of gamma rays. However, beta and positron radiation may also be detected, and are of particular relevance in PET imaging.

The sensor may be a room temperature, solid-state CdZnTe (CZT) detector, configured as a single-pixel or a multi-pixel detector. Alternatively, another solid-state detector such as CdTe, HgI, Si, Ge, or the like, or a scintillation detector, such as NaI(TI), LSO, GSO, CsI, CaF, or the like, or a combination of scintillation materials and photodiode arrays may be used.

Two technologies of computed tomography for radioactive emission are known.

i. Single photon emission computed tomography (SPECT), in which single radioactive emission events are detected, around a body. The detection of a large number of photons may be used to form a three-dimensional functional image and thus identify the source of the radiation.

ii. Positron emission tomography (PET), in which a positron is emitted from the radioactive isotope. Upon its interaction with an electron, annihilation occurs, and the two photons produced by the annihilation travel in opposite directions. Their detection by coincidence counting identifies an exact path upon which the annihilation took place. Again, the detection of a large number of photons may be used to form a three-dimensional functional image and identify the source of the radiation, especially using the fact that in PET, the photon paths for coincidence counts are known,

Attenuation by the surrounding tissue introduces a certain error.

Various radiopharmaceuticals can be synthesized to target specific molecules present in the target tissue cells. For example, [¹⁸F] FDG (fluorodeoxyglucose), or antibody fragment labeled with [⁶⁴Cu]. Others may be found in http://www.crump.ucla.edu/software/lpp/radioisotopes/tracers.html. Additional details and descriptions may be found in Breast Cancer Res. 2001, 3:28-35.

Tissue Characterization by Temperature Imaging: Temperature Imaging for locating and detecting neoplastic tissue has been known, since the 1950's, when it was discovered that the surface temperature of skin in the area of a malignant tumor exhibited a higher temperature than that expected of healthy tissue. Thus, by measuring body skin temperatures, it became possible to screen for the existence of abnormal body activity such as cancerous tumor growth. With the development of liquid crystals and methods of forming temperature responsive chemical substrates, contact thermometry became a reality along with its use in medical applications. Devices employing contact thermometry could sense and display temperature changes through indicators, which changed colors, either permanently or temporarily, when placed in direct physical contact with a surface such as skin, reflecting a temperature at or near the point of contact. An abnormal reading would alert a user to the need for closer, more detailed examination of the region in question. However, the art in this area has been directed primarily at sensing and displaying temperatures on exterior skin surfaces.

U.S. Pat. No. 3,830,224, to Vanzetti et al., whose disclosure is incorporated herein by reference, disclosed the placement of temperature responsive, color changing liquid crystals at various points in a brassiere for the purpose of detecting the existence of breast cancer.

US Patent RE 32,000, to Sagi, entitled, “Device for Use in Early Detection of Breast Cancer,” whose disclosure is incorporated herein by reference, disclosed a device comprising a flexible, heat-conductive web, preferably in the form of a disc-shaped patch having an adhesive layer on one side thereof and a peelable layer removably secured thereto by the adhesive layer. On the other side thereof, the device comprises an array of spaced-apart indicators, each of the indicators comprising a dye or a pigment and a temperature sensitive substance (crystalline organic chemical) which melts at a relatively precise temperature which is approximately 0.5.degree. F. different from the adjacent indicator. As many indicators are used as are necessary to cover the desired temperature range. The device is incorporated into the breast-receiving cups of a brassiere and mirror image quadrants of the two breasts are scanned and the device is visually examined to determine the number of indicators which have displayed a change in color, thus apprising the person of the existence of abnormality in the mammary tissue.

U.S. Pat. No. 6,135,968, to Brounstein, entitled, “Differential temperature measuring device and method”, whose disclosure is incorporated herein by reference, describes a device and method for sensing temperatures at internal body locations non-surgically accessible only through body orifices. The device is particularly useful in medical applications such as screening for cancer and other abnormal biological activity signaled by an increase in temperature at a selected site. As applied to prostate examinations, the device is temporarily, adhesively affixed to a user's fingertip or to a mechanical probe. In the preferred embodiment, the device includes two temperature-sensing elements, which may include a plurality of chemical indicators. Each indicator changes color in response to detection of a predetermined particular temperature. When properly aligned and installed, the first element is located on the palmar surface of the fingertip while the second element is located on the dorsal surface of the fingertip. After an examination glove has been donned over the fingertip carrying the device, a prostate examination is performed during which the first element is brought into constant but brief contact with the prostate region and the second element is similarly, simultaneously brought into contact with a dermal surface opposing the prostate region. Upon withdrawal of the fingertip from the rectum and removal of the glove, the two temperature sensing elements may be visually examined in order to determine the temperatures detected by each one. A significant difference in observed temperatures indicates the possibility of abnormal biological activity and the need for further diagnostic or medical procedures.

Tissue Characterization using Biosensors: Biosensors may be of catalytic type that integrated enzymes, cellular organelles, tissues or whole microorganisms with transducers that convert a biological response into a digital electronic signal. The principal transducers used are electrochemical, optical, or thermometric. Biosensors may also be of affinity type. Affinity biosensors deliver information about the binding of antibodies to antigens, cell receptors to their ligands, and DNA and RNA to nucleic acid with a complementary sequence. Still, additional types are fully integrated biochip devices that perform as micro bio-reactors. All types can be used in high-density arrays of bio-molecular sensors.

Some of these sensors are further discussed in:

(i) Enzyme and Microbial Biosensors: Techniques and Protocols. Ed. A. Mulchandani & K. R. Rogers (Humana Press, 1998);

(ii) Affinity Biosensors: Techniques and Protocols. Ed. A. Mulchandani & K. R. Rogers (Humana Press, 1998);

(iii) Journal: Biosensors & Bioelectronics:
a. Volume 20, Issue 8, Pages 1459-1695 (15 Feb. 2005);
b. Volume 20, Issue 6, Pages 1029-1259 (15 Dec. 2004);
c. Volume 20, Issue 5, Pages 917-1028 (15 Nov. 2004);
d. Volume 20, Issue 1, Pages 1-142 (30 Jul. 2004);
e. Volume 20, Issue 12, Pages 2387-2593 (15 Jun. 2005);

(iv) Journal: Sensors & actuators B (chemical). Volume:
a. Volume 103, Issues 1-2, Pages 1-473 (29 Sep. 2004);
b. Volume 102, Issue 1, Pages 1-177 (September 2004); and
c. Volume 106, Issue 1, Pages 1-488 (29 Apr. 2005).

Tissue Characterization using chemical sensors: Chemical sensors detect the presence of various types of chemical compounds and states. For example, ions, such as, but not limited to, Na, K; dissolved gases, such as, but not limited to, Oxygen, carbon di-oxide; and Ph of solution.

Some of these sensors are further discussed in:

(i) Sensors: A comprehensive survey. Volume 2: Chemical and biochemical sensors, Part I. Ed. W. Gopel, J. Hesse, & J. N. Zenel (VCH, 1991)

(ii) Sensors: A comprehensive survey. Volume 3: Chemical and biochemical sensors, Part II. Ed. W. Gopel, J. Hesse, & J. N. Zenel (VCH, 1992)

(iii) Journal: Sensors & actuators B (chemical). Volume:
a. Volume 103, Issues 1-2, Pages 1-473 (29 Sep. 2004);
b. Volume 102, Issue 1, Pages 1-177 (September 2004);
c. Volume 106, Issue 1, Pages 1-488 (29 Apr. 2005); and
d. Volume 108, Issues 1-2, Pages 1-1000 (22 Jul. 2005).

Tissue Characterization using Mechanical Sensors: Mechanical sensors measure a physical property of the tissue in contact with the sensor. One example of a mechanical sensor uses, tactile sensing that measures the pressure sensed on the sensor surface. An optical tactile sensor having a transparent elastic tactile portion has been taught in U.S. Pat. No. 6,909,084 to Tachi and Kajimoto, whose disclosure is incorporated herein by reference, provides an optical tactile sensor with a tactile section and imaging means, the tactile section comprising a transparent elastic body and a plurality of groups of markers provided inside the elastic body, each marker group made up of a number of colored markers, with markers making up different marker groups having different colors for each group, and behavior of the colored markers when an object touches the elastic body being photographed by the imaging means. Preferably the marker groups have mutually different spatial arrangements. Furthermore, mechanical sensors are discussed in: Sensors: A comprehensive survey. Volume 7: Mechanical sensors. Ed. W. Gopel, J. Hesse, & J. N. Zenel (VCH, 1994).

It will be appreciated that the device, in accordance with embodiments of the present invention, may adapted for human tissue and (or) for animal tissue.

It is expected that during the life of this patent many relevant ergonomic devices for tissue characterization will be developed and the scope of the term ergonomic devices for tissue characterization is intended to include all such new technologies a priori.

As used herein the term “about” and “substantially” refer to ±20%.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, any citation or identification of any reference in this application shall not be construed as an admission that such reference is available as prior art to the present invention.

What is claimed is:

1. An ergonomic device for intraoperative tissue characterization, comprising:
a body, which comprises:
proximal and distal portions, with respect to the tissue, as it is characterized;
a gripping handle, at the distal portion; and
a sensor head, at the proximal portion, the sensor head comprising at least one sensor for tissue characterization,
wherein the gripping handle and the sensor head are arranged at an angle α, wherein in absolute value, α>10 degrees.

2. The ergonomic device of claim 1, wherein the angle α between the gripping handle and the sensor head is adjustable.

3. The ergonomic device of claim 1, wherein the angle α between the gripping handle and the sensor head is adjustable in two planes.

4. The ergonomic device of claim 1, wherein the angle α between the gripping handle and the sensor head is adjustable in three planes.

5. The ergonomic device of claim 1, wherein the sensor head further includes a vacuum system, for improved contact between a sensing surface and the tissue to be characterized.

6. The ergonomic device of claim 1, wherein the sensor head further includes a light fixture, configured for lighting the tissue, as it is characterized.

7. The ergonomic device of claim 1, wherein the sensor head further includes a marking module, configured for marking the tissue, as it is characterized.

8. The ergonomic device of claim 1, wherein the sensor head further includes a transparent frame, which surrounds the at least one sensor and enables an operator to observe the tissue, as it is characterized, through it.

9. The device of claim 1, wherein the at least one sensor is an irradiative sensor, selected from the group consisting of an optical sensor, an X-ray sensor, an RF sensor, a MW sensor, an infrared thermography sensor, and an ultrasound sensor.

10. The device of claim 1, wherein the at least one sensor is selected from the group consisting of an MR sensor, an impedance sensor, a temperature sensor, a biosensor, a chemical sensor, a radioactive-emission sensor, a nonirradiative RF sensor, and a mechanical sensor.

11. The device of claim 1, and further comprising a plurality of sensors.

12. The device of claim 1, wherein the at least one sensor includes at least two different types of sensors.

13. The device of claim 1, wherein the at least one sensor includes at least two different types of sensors, selected from the group consisting of optical sensors, X-ray sensors, RF sensors, MW sensors, infrared thermography sensors, ultrasound sensors, MR sensors, impedance sensors, temperature sensors, biosensors, chemical sensors, radioactive-emission sensors, mechanical sensors, and nonirradiative RF sensors.

14. An ergonomic device for intraoperative tissue characterization, comprising:
a body, which comprises:
proximal and distal portions, with respect to the tissue, as it is characterized;
a gripping handle, at the distal portion; and
a sensor head, at the proximal portion, the sensor head comprising:
at least one sensor for tissue characterization; and
a light fixture, configured for lighting the tissue, as it is characterized.

15. The ergonomic device of claim 14, wherein the gripping handle and the sensor head are arranged at an angle α, wherein in absolute value, α>10 degrees.

16. The ergonomic device of claim 15, wherein the angle α between the gripping handle and the sensor head is adjustable.

17. The ergonomic device of claim 15, wherein the angle α between the gripping handle and the sensor head is adjustable in two planes.

18. The ergonomic device of claim 15, wherein the angle α between the gripping handle and the sensor head is adjustable in three planes.

19. The ergonomic device of claim 14, wherein the sensor head further includes a vacuum system, for improved contact between a sensing surface and the tissue to be characterized.

20. The ergonomic device of claim 14, wherein the sensor head further includes a marking module, configured for marking the tissue, as it is characterized.

21. The ergonomic device of claim 14, wherein the sensor head further includes a transparent frame, which surrounds the at least one sensor and enables an operator to observe the tissue, as it is characterized, through it.

22. The device of claim 14, wherein the at least one sensor is an irradiative sensor, selected from the group consisting of an optical sensor, an X-ray sensor, an RF sensor, a MW sensor, an infrared thermography sensor, and an ultrasound sensor.

23. The device of claim 14, wherein the at least one sensor is selected from the group consisting of an MR sensor, an impedance sensor, a temperature sensor, a biosensor, a chemical sensor, a radioactive-emission sensor, a nonirradiative RF sensor, and a mechanical sensor.

24. The device of claim 14, and further comprising a plurality of sensors.

25. The device of claim 14, wherein the at least one sensor includes at least two different types of sensors.

26. The device of claim 14, wherein the at least one sensor includes at least two different types of sensors, selected from the group consisting of optical sensors, X-ray sensors, RF sensors, MW sensors, infrared thermography sensors, ultrasound sensors, MR sensors, impedance sensors, temperature sensors, biosensors, chemical sensors, radioactive-emission sensors, mechanical sensors, and nonirradiative RF sensors.

27. An ergonomic device for intraoperative tissue characterization, comprising:
a body, which comprises:
proximal and distal portions, with respect to the tissue, as it is characterized;
a gripping handle, at the distal portion; and
a sensor head, at the proximal portion, the sensor head comprising:
at least one sensor for tissue characterization; and
a marking module, configured for marking the tissue, as it is characterized.

28. The ergonomic device of claim 27, wherein the gripping handle and the sensor head are arranged at an angle α x, wherein in absolute value, α>10 degrees.

29. The ergonomic device of claim 28, wherein the angle α between the gripping handle and the sensor head is adjustable.

30. The ergonomic device of claim 28, wherein the angle α between the gripping handle and the sensor head is adjustable in two planes.

31. The ergonomic device of claim 28, wherein the angle α between the gripping handle and the sensor head is adjustable in three planes.

32. The ergonomic device of claim 27, wherein the sensor head further includes a vacuum system, for improved contact between a sensing surface and the tissue to be characterized.

33. The ergonomic device of claim 27, wherein the sensor head further includes a light fixture, configured for lighting the tissue, as it is characterized.

34. The ergonomic device of claim 27, wherein the sensor head further includes a transparent frame, which surrounds the at least one sensor and enables an operator to observe the tissue, as it is characterized, through it.

35. The device of claim 27, wherein the at least one sensor is an irradiative sensor, selected from the group consisting of an optical sensor, an X-ray sensor, an RF sensor, a MW sensor, an infrared thermography sensor, and an ultrasound sensor.

36. The device of claim 27, wherein the at least one sensor is selected from the group consisting of an MR sensor, an impedance sensor, a temperature sensor, a biosensor, a chemical sensor, a radioactive-emission sensor, a nonirradiative RF sensor, and a mechanical sensor.

37. The device of claim 27, and further comprising a plurality of sensors.

38. The device of claim 27, wherein the at least one sensor includes at least two different types of sensors.

39. The device of claim 27, wherein the at least one sensor includes at least two different types of sensors, selected from the group consisting of optical sensors, X-ray sensors, RF sensors, MW sensors, infrared thermography sensors, ultrasound sensors, MR sensors, impedance sensors, temperature sensors, biosensors, chemical sensors, radioactive-emission sensors, mechanical sensors, and nonirradiative RF sensors.

40. An ergonomic device for intraoperative tissue characterization, comprising:
a body, which comprises:
proximal and distal portions, with respect to the tissue, as it is characterized;
a gripping handle, at the distal portion; and
a sensor head, at the proximal portion, the sensor head comprising:
at least one sensor for tissue characterization; and
a transparent frame, which surrounds the at least one sensor and enables an operator to observe the tissue, as it is characterized, through it.

41. The ergonomic device of claim 40, wherein the gripping handle and the sensor head are arranged at an angle α, wherein in absolute value, α>10 degrees.

42. The ergonomic device of claim 41, wherein the angle α between the gripping handle and the sensor head is adjustable.

43. The ergonomic device of claim 41, wherein the angle α between the gripping handle and the sensor head is adjustable in two planes.

44. The ergonomic device of claim 41, wherein the angle α between the gripping handle and the sensor head is adjustable in three planes.

45. The ergonomic device of claim 40, wherein the sensor head further includes a vacuum system, for improved contact between a sensing surface and the tissue to be characterized.

46. The ergonomic device of claim 40, wherein the sensor head further includes a light fixture, configured for lighting the tissue, as it is characterized.

47. The ergonomic device of claim 40, wherein the sensor head further includes a marking module, configured for marking the tissue, as it is characterized.

48. The device of claim 40, wherein the at least one sensor is an irradiative sensor, selected from the group consisting of an optical sensor, an X-ray sensor, an RF sensor, a MW sensor, an infrared thermography sensor, and an ultrasound sensor.

49. The device of claim 40, wherein the at least one sensor is selected from the group consisting of an MR sensor, an impedance sensor, a temperature sensor, a biosensor, a chemical sensor, a radioactive-emission sensor, a nonirradiative RF sensor, and a mechanical sensor.

50. The device of claim 40, and further comprising a plurality of sensors.

51. The device of claim 40, wherein the at least one sensor includes at least two different types of sensors.

52. The device of claim 40, wherein the at least one sensor includes at least two different types of sensors, selected from the group consisting of optical sensors, X-ray sensors, RF sensors, MW sensors, infrared thermography sensors, ultrasound sensors, MR sensors, impedance sensors, temperature sensors, biosensors, chemical sensors, radioactive-emission sensors, mechanical sensors, and nonirradiative RF sensors.
